(19) United States
(12) Patent Application Publication
(10) Pub. No.: US 2011/0143960 A1
(43) Pub. Date: Jun. 16, 2011

(54) 3D-MODELS FOR HIGH-THROUGHPUT SCREENING DRUG DISCOVERY AND DEVELOPMENT

(76) Inventor: Daniel V. LaBarbera, Centennial, CO (US)

(21) Appl. No.: 12/965,747

(22) Filed: Dec. 10, 2010

Related U.S. Application Data
(60) Provisional application No. 61/285,539, filed on Dec. 10, 2009.

(51) Int. Cl. C40B 30/06; C12Q 1/02

(52) U.S. Cl. 506/10; 435/29

(57) ABSTRACT

The invention provides high throughput screening methodologies for identifying agents that can modulate Epithelial-Mesenchymal Transition (EMT) and/or Mesenchymal-Epithelial Transition (MET) phenotypes of a cell, uses of such agents and methods of identifying a patient that is likely to respond or unlikely to respond to treatment with such agents.

### CROSS REFERENCE TO RELATED APPLICATION

This application claims the benefit of priority under 35 U.S.C. §119(e) to U.S. Provisional Patent Application Ser. No. 61/285,539 filed Dec. 10, 2009, which is incorporated herein by reference.

### TECHNICAL FIELD

The invention relates to drug discovery and development and particularly 3D models that may be used in high throughput screening techniques.

### BACKGROUND OF INVENTION

In 1968 Elizabeth D. Hay first described the concept that epithelial cells undergo a phenotypic change to become motile or mesenchymal (Hay, E. D., Organization and fine structure of epithelium and mesenchyme in the developing chick embryo. In Epithelial-Mesenchymal Interactions, Fleischmajer, R.; Billingham, R. E., Eds. Williams & Wilkins: Baltimore, 1968; pp 31-55). This reversible transformation now described as Epithelial-Mesenchymal Transition (EMT) has been shown to be an important mechanism in human biological processes including: embryonic development, wound healing, and cancer progression and metastasis. A recent review series on EMT, prompted by the EMT research community, has further categorized EMT into three types including: embryonic development and organ formation (type 1), wound healing and fibrosis (type 2), and cancer progression and metastasis (type 3). As a result, type 3 EMT is emerging as a valid target for drug development in the treatment of cancer and fibrosis.

Currently, there remain gaps in our understanding of the signaling events that govern EMT, and the reversion, Mesenchymal-Epithelial Transition (MET). For example, in 1987 Nagafuchi et al. showed that exogenous expression of wild type E-cadherin into L-fibroblasts (devoid of E-cadherin) showed a morphological change resulting in the formation of tight juctions (Nagafuchi, A.; Shirayoshi, Y.; Okazaki, K.; Yasuda, K.; Takeichi, M., Transformation of cell adhesion properties by exogenously introduced E-cadherin cDNA. Nature 1987, 329, 341-3). Since this report, it has been well documented that when E-cadherin is exogenously expressed in mesenchymal cells, there is a phenotypic reversion resembling an epithelial phenotype (Hay, E. D., The mesenchymal cell, its role in the embryo, and the remarkable signaling mechanisms that create it. Dev Dyn 2005, 233, 706-20). More recently Wang et al. demonstrated that inhibiting signaling pathways such as Ras-MAPKinase, or PI3Kinase led to moderate inhibition of in vitro cell invasion with no morpholocial changes indicative of a phenotypic reversion (Wang, F.; Hansen, R. K.; Radisky, D.; Yoneda, T.; Barcellos-Hoff, M. H.; Petersen, O. W.; Turley, E. A.; Bissell, M. J., Phenotypic reversion or death of cancer cells by altering signaling pathways in three-dimensional contexts. J Natl Cancer Inst 2002, 94, 1494-503). However, this study did not measure the changes in expression of specific EMT markers.

Around the world approximately 1 million women are diagnosed with breast cancer each year. In the United States, the probability of developing invasive breast cancer in women is 1 in 8 from birth to death and there are about 180,000 (about 72%) out of 250,000 new cases of invasive breast cancer each year. The 5-year survival rate for women diagnosed with in situ or localized breast cancer is 98%, which decreases significantly for women diagnosed with invasive metastatic breast cancer to only 27%. Therefore, metastatic dissemination remains to be the primary cause of mortality in breast cancer patients. In particular, 15-20% of breast cancers display basal-like (EGFR, vimentin positive) and or triple negative (TN) (HR-negative and HER2-negative) phenotypes, which are associated with aggressive metastatic breast cancer, resistance to chemotherapy and an overall poor prognosis.

The pathogenesis of basal-like and TN breast cancers is still poorly understood and it remains unclear as to what mechanisms drive these tumor cells to proliferate and metastasize. As a result, there are no specific targeted therapeutic systemic regimens for the treatment of these types of cancer, which ultimately contributes to the overall poor prognosis. However, growing evidence indicates that these types of cancer undergo EMT resulting in metastasis (Sarrio, D.; Rodriguez-Pinilla, S. M.; Hardisson, D.; Cano, A.; Moreno-Bueno, G.; Palacio s, J., Epithelial-mesenchymal transition in breast cancer relates to the basal-like phenotype. Cancer Res. 2008, 68, 989-97).

In the modern era of tissue culture spanning the last five decades, scientists have been experimenting with 3D-culture systems including extra cellular matrices (ECM). Inevitably, 3D-culture models prove to be superior at recapitulating in vivo-like growth and differentiation of tissues as compared to 2D models. In the field of breast cancer metastasis, pioneering work by Mina Bissell's laboratory describing the reciprocal and dynamic relationship between tissue structure and function has transcended our current understanding of the tumor microenvironment and its role in tumor suppression and metastatic dissemination. A 1997 article by Weaver et al. exemplified the importance of the microenvironment on EMT and the malignant phenotype (Weaver, V. M.; Petersen, O. W.; Wang, F.; Larabell, C. A.; Briand, P.; Damsky, C.; Bissell, M. J., Reversion of the malignant phenotype of human breast cells in three-dimensional culture and in vivo by integrin blocking antibodies. J Cell Biol 1997, 137, 231-45). This study demonstrated that inhibiting integrin signaling (blocking extracellular matrix-integrin interactions) with antibodies lead to a reversion of the malignant phenotype in 3D-tissue culture. This phenotypic reversion was not observed in cells grown in a monolayer, demonstrating the importance of the microenvironment.

A major barrier in the use of 3D-spheroid models incorporating ECM for high-throughput drug screening has been the cost and complexity in uniform miniaturization with reproducible standardized protocols. Methods describing the standardized 3D-culture of breast carcinoma tumor spheroids in ECM in laminin/collagen proteins (MATRIGEL™) have been published. Although these methods are suitable to model breast cancer and EMT in vitro they do not allow for the uniform culture of single spheroids, which is a crucial parameter when developing spheroid models for HTS. Recent methods have been published describing the culture of uniform single spheroids using a liquid-overlay technique in 96 well plates coated with agarose (no MATRIGEL™) or poly-HEMA. Agarose acts as a non-adherent concave surface, which allows cells to aggregate, facilitating spheroid formation. While this methodology allows for the successful culture of spheroids of various types of cancer, it relies on the inherent ability of the cell type of interest to differentiate into spheroids independent of the ECM. Furthermore, many carcinomas cannot differentiate into tumor spheroids independently of the ECM, particularly the highly basal-like and mesenchymal cells. Each of the foregoing disadvantages is overcome by the testing apparatus and method of this invention.

### SUMMARY OF INVENTION

The present invention provides methods of screening potential therapeutic agents for activity in modulating the Epithelial-Mesenchymal Transition (EMT) phenotype of a cell by establishing at least one three-dimensional (3D) spheroid of cells in individual wells of a multi-welled plate that is capable of sustaining a tissue culture and contacting the cell spheroid(s) with the potential therapeutic agent. The cells in the spheroid(s) are assayed for expression or activity of a biomarker that is indicative of modulation of EMT activity of a cell. The cells used to form the spheroid are cells capable of sustained growth under tissue culture conditions and may be neoplastic cells, including tumor cells that have been isolated from a human, such as malignant tumor cells from a human breast cancer, lung cancer, prostate cancer, colon cancer, melanoma cancer, or cancer of the bone and connective tissues. The cells may be stem cells including embryonic stem cells or adult stem cells, progenitor cells, bone marrow stromal cells macrophages, fibroblast cells, endothelial cells, epithelial cells, or mesenchymal cells.

The cells forming the spheroid(s) are transformed with at least one heterologous nucleic acid molecule that encodes one or more biomarkers associated with the epithelial or mesenchymal phenotypes. In one embodiment, the heterologous nucleic acid molecule(s) are chromosomally integrated into the genome of the cells. In a preferred embodiment, the heterologous nucleic acid molecule encodes a biomarker linked to an indicator that can be detected in situ following expression of the biomarker. The indicator may be a compound that is readily detectable using a detection technique such as dark versus light detection, fluorescence or chemiluminescence spectrophotometry, scintillation spectroscopy, chromatography, liquid chromatography/mass spectroscopy (LC/MS), and colorimetry. The indicator compound may be a fluorogenic or fluorescent compound, chemiluminescent compound, calorimetric compound, UV/VIS absorbing compound, radionucleotide, Red Fluorescence Protein (RFP), Green Fluorescent Protein (GFP), luciferase, or a combination of these. Exemplary biomarkers include Epithelial (E)-cadherin, Zinc finger E-box binding homeobox 1 (ZEB1), and Vimentin.

The multi-welled plate is preferably a 96-well or a 1536-well cell culture plate. In one embodiment, the multi-welled plate is coated with a mixture of structural proteins including laminin and collagen (laminin/collagen proteins: MATRIGEL™, BD Biosciences). In one embodiment, the multi-welled plate is coated with agarose. In another embodiment, the multi-welled plate is coated with collagen and agarose.

Potential therapeutic agents may include a native or endogenous ligand or ligands, a biological sample suspected of containing a native or endogenous ligand or ligands, a combinatorial library of small molecules, hormones, antibodies, polysaccharides, anti-cancer agents, natural products, terrestrial products, marine natural products, a molecule that binds with high affinity to a biopolymer such as a protein, a nucleic acid, and a polysaccharide, a purified or isolated biological molecule such as a protein, a nucleic acid, a silencing RNA (siRNA), a micro RNA (miRNA), and a short hairpin RNA (shRNA).

Secondary assays may be used to further assess the effect of the potential therapeutic agent on modulation of the EMT or MET characteristics of the cells. In one aspect, the cells are dissociated from a spheroid and the potential therapeutic agent is tested for the effect on migration of cells dissociated from the cell spheroid(s) through a collagen-coated membrane, such as a PET membrane coated with MATRIGEL™ in a Boyden chamber.

Another embodiment is a method to identify a cancer patient who is predicted to benefit or not benefit from therapeutic administration of an inhibitor of EMT by establishing a three-dimensional (3D) spheroid of cells from a sample of tumor cells from the patient and assaying for a level of a biomarker associated with EMT, such as Epithelial (E)-cadherin, Zinc finger E-box binding homeobox 1 (ZEB1), and/or Vimentin. The expression or activity level of the biomarker in the tumor cell sample is compared to a control level of the biomarker that may include a control level of the biomarker that has been correlated with sensitivity to the inhibitor or a control level of the biomarker that has been correlated with resistance to the inhibitor. Patients are selected as being predicted to benefit from therapeutic administration of the inhibitor, if the level of the biomarker in the patient's tumor cells is statistically similar to or greater than the control level of the biomarker that has been correlated with sensitivity to the inhibitor, or if the level of the biomarker in the patient's tumor cells is statistically greater than the level of the biomarker that has been correlated with resistance to the inhibitor. Patients are selected as being predicted to not benefit from therapeutic administration of the inhibitor, if the level of the biomarker in the patient's tumor cells is statistically less than the control level of the biomarker that has been correlated with sensitivity to the inhibitor, or if the level of the biomarker in the patient's tumor cells is statistically similar to or less than the level of the biomarker that has been correlated with resistance to the inhibitor.

### BRIEF DESCRIPTION OF DRAWINGS

FIG. 1, shows the MDA-MB-231 spheroid culture methodology suitable for high throughput screening. A) MDA-MB-231 cell aggregation on 96-well plates coated with agarose (top panel). When aggregated cells are coated with 5% MATRIGEL™ (laminin/collagen proteins) robust spheroid formation occurs resulting in spheroid diameters of 500 μm (day 6) and 1 mm (day 12) (bottom panel). B) Digital camera view depicting uniform single spheroids in a 96-well plate. C) Spheroid growth kinetics over 32 days. D) MDA-MB-231 spheroids cultured in 1536-well plates.

FIG. 2, shows A) The known signaling pathways that control or modulate EMT including small molecule inhibitors, and, B) Live spheroid vimentin expression after treatment with control modulators of EMT for 72 h. C) Spheroid viability measured by multiplexing the APH assay directly after measuring vimentin expression. The t-test analysis showed no statistical difference between spheroids treated with control EMT modulators and untreated spheroids.

FIG. 3 shows A) QPCR (normalized to untreated), and B) Corresponding western blot data after treatment with known antitumor agents.

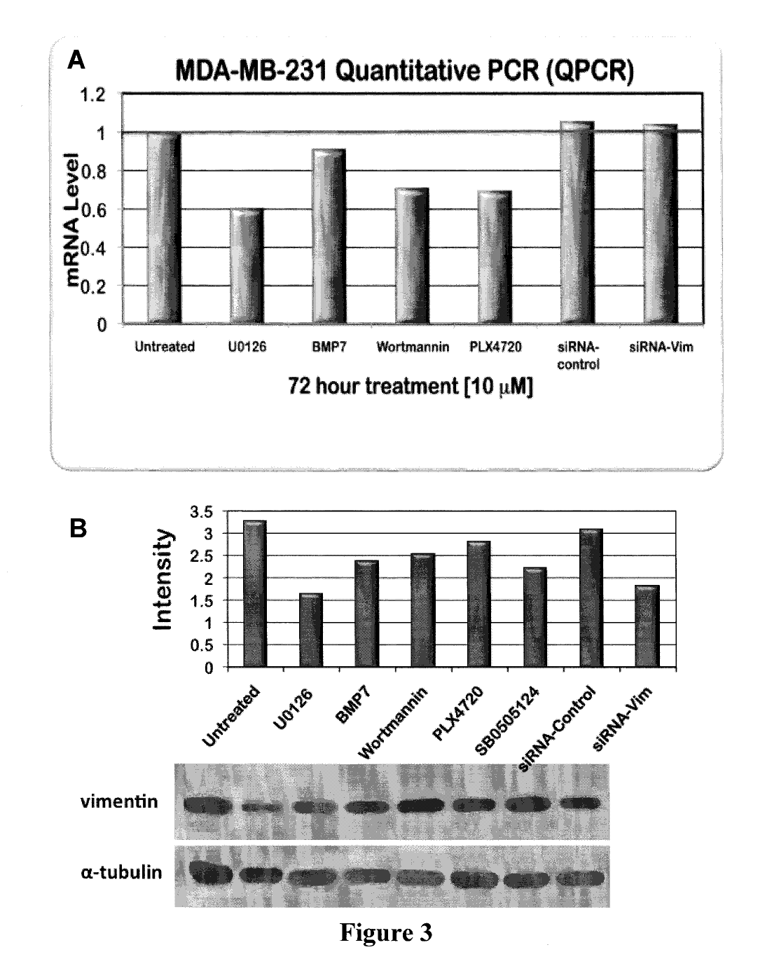

FIG. 4 shows the reversion of EMT characterized by biomarker expression. A) Western blot analysis of MDA-MB-231 cells treated with miRNA-200C. B) Western blot analysis after small molecule treatment resulting in downregulation of ZEB1. C) Western blot analysis of MDA-MB-231 cells treated with a therapeutic agent discovered using aspects of the proposed invention. D) Cell invasion assay depicting significant loss of invasive potential of MDA-MB-231 cells after treatment with therapeutic agent identified using methodology of the present invention.

FIG. 5 shows A) Live 3D-spheroid assay measuring vimentin expression followed by multiplexing cell viability with the APH assay. B) Comparison of activity normalized to untreated cells.

FIG. 6 shows the secondary assay development. The invasive potential of MDA-MB-231 spheroids was measured using modified Boyden chambers coated with laminin/collagen proteins (MATRIGEL™). Invading cells were fixed, stained with DAPI, and quantified by fluorescence microscopy using five random fields per filter insert in triplicate. U0126, PF2341066, Axitinib, and PKC412 inhibited the invasive potential of MDA-MB-231 spheroids by about 90% as compared to untreated spheroids (UT), *** P≦0.001. IGF1R and Dasatinib displayed no statistical difference as compared to UT MDA-MB-231 spheroids.

FIG. 7 shows Vimentin expression in live breast cancer cells (MDA-MB-231) growing as cell spheroids in 96-well plates following treatment with 11 therapeutic agents.

FIG. 8 shows Vimentin gene expression in A) VimPro-Fluc spheroids after 72 h treatment with control modulators of EMT normalized to spheroid viability and compared to vimentin protein expression using Western blot analysis. B) Dose response curves for both U0126 and Axitinib control modulators of EMT.

FIG. 9 shows the results of the screen of marine natural products. A) Vimentin expression results from primary screening of 230 marine secondary metabolites. The dashed line represents the hit limit calculated as three standard deviations away from the mean. B) The Z′ statistical validation per plate screened is indicated and the dashed black line represents the Z′ limit. C) The vimentin expression of 5 identified hits rescreened in triplicate D) Spheroid viability of five hits identified from primary screening. With the exception of lissoclinotoxin E (P≦0.001) all the hits identified showed no statistical difference in spheroid viability as compared to untreated spheroids on day 5. Spheroids treated with 10% Triton X-100 2 h prior to APH analysis represent a control that significantly induces spheroid death. E) Secondary assay screening confirmed the hits identified also inhibit the invasive potential of MDA-MB-231 spheroids by about 90% (P≦0.001) as compared to untreated day 8 spheroids.

This Summary of the Invention is neither intended nor should it be construed as being representative of the full extent and scope of the present invention. Moreover, references made herein to “the present invention,” or aspects thereof, should be understood to mean certain embodiments of the present invention and should not necessarily be construed as limiting all embodiments to a particular description. The present invention is set forth in various levels of detail in the Summary of the Invention as well as in the attached drawings and the Description of Embodiments and no limitation as to the scope of the present invention is intended by either the inclusion or non-inclusion of elements, components, etc. in this Summary of the Invention. Additional aspects of the present invention will become more readily apparent from the Description of Embodiments, particularly when taken together with the drawings.

### DESCRIPTION OF EMBODIMENTS

The present invention is drawn to an apparatus and method of efficient high-throughput identification of novel small molecule modulators of EMT that may be used as molecular tools or probes to further study the mechanisms and molecular targets of EMT.

The methodology of the present invention relies on a biomarker signature of Epithelial-Mesenchymal Transition (EMT) engineered to function as a readout to monitor this transition. Incorporating this biomarker signature readout in 3D-spheroid models adds an extra dimension of information to this screening technique, including microenvironment interactions and localization of these critical biomarkers.

One aspect of the invention is a method of screening compounds for activity in modulating the EMT phenotype of a cell. The method includes establishing at least one three-dimensional (3D) spheroid of cells in individual wells of a multi-welled plate that is capable of sustaining a tissue culture. The cell spheroid(s) are contacted with a potential therapeutic agent and assayed for a marker indicative of modulation of EMT activity.

The cells which form the cell spheroid are cells capable of sustained growth under tissue culture conditions. In one embodiment, the cells are neoplastic cells that may include isolated cells from solid tumors including carcinomas or sarcomas and cancers of the circulating cells including leukemias and lymphomas. In a preferred embodiment, the cells are tumor cells that have been isolated from a human. In a particularly preferred embodiment, the cells are human malignant tumor cells, such as, breast cancer, lung cancer, prostate cancer, colon cancer, melanoma cancer, and cancer of the bone and connective tissues. In addition, cells that are useful in the screening methodology of the present invention may include stem cells such as embryonic stem cells or adult stem cells, progenitor cells, bone marrow stromal cells, macrophages, fibroblast cells, endothelial cells, epithelial cells, and mesenchymal cells.

The multi-welled plate may be a 96- or 384-well cell culture plate. Preferably the multi-welled plate is a 1536-well cell culture plate. In one embodiment the multi-welled plate is coated with laminin/collagen proteins (MATRIGEL™), which acts as a substrate in which to grow spheroids and replicate the tumor microenvironment. In another embodiment, the multi-welled plate has a coating of agarose, which provides a non-adherent surface, facilitating tumor cell aggregation. In a preferred embodiment the multi-welled plate has a surface coated with both collagen IV basement membrane and agarose to induce spheroid formation in a way that is not achieved with agarose alone.

A potential therapeutic agent is a substance which can modulate the EMT and/or MET in the cells by binding or other intermolecular interaction with cellular proteins. By “modulate” is intended an increase, decrease, or other alteration of any or all biological activities or properties associated with EMT and/or MET. Thus, a native or endogenous ligand or ligands is a “potential therapeutic agent.” A biological sample suspected of containing a native or endogenous ligand or ligands is also a “potential therapeutic agent.” Small molecules and combinatorial libraries of small molecules are also “potential therapeutic agents.” A candidate substance identified according to a screening assay described herein has the ability to modulate MET and/or EMT biological activity. Such a candidate substance has utility in the treatment of disorders and conditions wherein modulation of EMT and/or MET is desirable, as well as in the purification and screening methods disclosed herein. In one embodiment, the potential therapeutic agent is a chemical synthesized entirely from an organic synthesis scheme, a “small molecule” that binds with high affinity to a biopolymer such as protein, nucleic acid, or polysaccharide and in addition alters the activity or function of the biopolymer. In another embodiment, the potential therapeutic agent is an isolated or purified biological molecule including small molecules, protein, nucleic acid (DNA or RNA), silencing RNA (siRNA), micro RNA (miRNA), short hairpin RNA (shRNA), a hormone (such as a growth factor), an antibody (or an antibody fragment) or polysaccharide. In another embodiment the potential therapeutic agent is a known anti-cancer agent used in clinical practice. Therapeutic agents useful in the screening assays of the present invention include terrestrial natural products, marine natural products, proteins or other transcription-modifying agents that are suspected of altering the EMT or MET phenotype and or inhibition of invasion, migration or metastasis.

In one embodiment, the cells forming the spheroid(s) of cells, are transformed with at least one heterologous nucleic acid molecule that encodes one or more biomarkers associated with the epithelial or mesenchymal phenotypes. In a preferred embodiment, the recombinant nucleic acid molecule(s) are chromosomally integrated into the genome of the cell. Preferably the biomarkers are linked to an indicator that can be detected in situ following expression of the biomarker. The term “indicator” is meant to refer to a chemical species or compound that is readily detectable using a standard detection technique, such as dark versus light detection, fluorescence or chemiluminescence spectrophotometry, scintillation spectroscopy, chromatography, liquid chromatography/mass spectroscopy (LC/MS), colorimetry, and the like. Representative indicator compounds thus include, but are not limited to, fluorogenic or fluorescent compounds, chemiluminescent compounds, calorimetric compounds, UV/VIS absorbing compounds, radionucleotides and combinations thereof. Exemplary indicators for use in the screening methods of the present invention are proteins including Red Fluorescence Protein (RFP), which fluoresces when exposed to light of wavelength 558 nm, Green Fluorescent Protein (GFP) which fluoresces when exposed to light of wavelength 395 nm, and luciferase, which produces light in the conversion of luciferin and oxygen to oxyluciferin. These indicators, when coupled with an automated plate reading mechanism, form an embodiment of the present invention that is readily amenable to both robotic and very high throughput systems.

Biomarkers useful for the screening assays of the present invention include proteins, the expression of which is modified when the cell(s) undergoes EMT or MET. EMT is characterized by loss of expression of epithelial markers, such as epithelial (E)-cadherin and cytokeratins, accompanied by the increased expression of mesenchymal markers, such as neuronal (N)-cadherin and vimentin. For this reason, a collection of markers may also be used in the cells of the screening assay in order to observe and investigate changes associated with EMT. Particularly robust screening assays of the present invention therefore include adherence, transcription factor, and cytoskeleton markers. Particularly useful biomarkers include:
Epithelial (E)-cadherin (adherence marker): Greater than 80% of life threatening tumors are of the carcinoma type derived from epithelial tissues. E-cadherin-mediated cell-cell adhesion is typically lost in malignant tumor progression and there is an inverse correlation between E-cadherin levels, tumour grade and patient mortality rates. As a result, E-cadherin expression is generally accepted as a classical marker for EMT and metastasis both in vitro and in vivo. In particular, aberrant expression of E-cadherin in breast cancer is associated with the more aggressive ER-negative and basal-like phenotypes. Exogenous expression of E-cadherin results in a partial phenotypic switch from mesenchymal to epithelial transition (MET) in MDA-MB-231 cells leading to distinct morphological changes and inhibition of cell invasion.
Zinc finger E-box binding homeobox 1 (ZEB1) (transcription marker): There are a number of transcription factors that regulate EMT through suppression of E-cadherin. These include among others, snail1/2, twist, TCF3/E47, and ZEB1/2. However, recent reports have shown that high endogenous levels of ZEB1 correlate with the basal-like phenotype. ZEB1 has been shown to be a master regulator of EMT by modulating the expression of proteins involved in cell polarity, adhesion, apoptosis, and basement membrane components. Importantly, ZEB1 expression has been shown to be inversely correlated with E-cadherin expression. Silencing of ZEB1 in MDA-MB-231 cells restores E-cadherin expression leading to a partial restoration of the epithelial phenotype. More recently, the microRNA 200 family (miR-200) have been shown to suppress EMT by inhibiting ZEB1 expression therefore maintaining E-cadherin expression and epithelial homeostasis. However, aberrant signaling by TGF-β/BMP/Smad has been shown to enhance the expression of ZEB1 leading to a double-negative feedback loop between ZEB1/2 and the miR-200 family members repressing promoter activity and expression. Thus, ZEB1 repression of miR-200 leads to stabilized EMT and mesenchymal homeostasis.
Vimentin (cytoskeleton marker): Importantly, basal-like/TN mesenchymal cells are characterized by induced vimentin expression, which is the highest expressed EMT marker in this type of breast cancer. When vimentin expression is suppressed in these cell types the inhibition of carcinoma cell migration and invasion is observed. This demonstrates that vimentin expression not only correlates with the mesenchymal phenotype but also plays a functional role in EMT and migration. Important cellular functions of vimentin include the modulation of cell adhesion by regulating integrin function and vesicle trafficking, providing scaffolding for protein kinases such as Raf-1 and ERK, regulation of DNA transcription and repair, and participation in the regulation of apoptosis through sequestering interactions with proteins, such as p53, caspases, and 14-3-3. Vimentin is a proven marker for EMT and the invasive phenotype. Its high expression in basal-like breast cancer is associated with metastatic dissemination, resistance to chemotherapy and an overall poor prognosis.

The use of a biomarker linked to an indicator protein provides the capability of high throughput screening (HTS) when coupled with an automated means of detecting the expression of the biomarker(s). Thus, in a preferred embodiment of the invention the detecting step includes means for automated detection of the one or more biomarkers associated with EMT or MET. In one embodiment of the screening methods of the present invention, the automated detection means is engineered as a readout to monitor EMT/MET in the cell spheroids. In a preferred embodiment, this “EMT signature” will include E-cadherin (RFP-fusion protein), ZEB1 (promoter-luciferase reporter), and vimentin (GFP-fusion protein).

### Secondary Assays

In another embodiment of the invention, a counter assay is used to measure cell viability and this assay is multiplexed with the EMT biomarker signature readout in the screening assay described above. In this embodiment, the screening assay described above is multiplexed with another assay optimized to measure the inhibition of cell invasion and migration. A preferred embodiment of such secondary assays includes the use of the Boyden chamber methodology, preferably formatted to 96 well plates (Guan, J.-L., Cell migration: developmental methods and protocols. Humana Press: Totowa, N.J., 2005). This assay is used to confirm hits and generate lead compounds for optimization as molecular probes.

Compounds discovered using the screening methodologies of this invention can be developed into therapeutics that systemically target the inhibition of metastasis. Additionally, these techniques can be adapted to conduct individualized treatment on patients in the clinical setting. For example, primary breast carcinoma cells isolated from patients may be cultured in the 3D screening systems of the present invention and screened against known lead compounds from previous chemical library screenings as well as clinical agents in use today in order to identify patients that are likely or unlikely to respond to one or more agents available to the treating physician.

Thus, one embodiment of the invention is a therapeutic agent identified as effectively modifying MET and/or EMT in the screening methodologies of the present invention. Another related embodiment of the invention relates to the use of such therapeutic entities to treat a cancer patient, either alone, or in combination with other anti-cancer therapies. Another related embodiment of the invention is the preparation of a medicament for the treatment of cancer wherein the medicament includes a therapeutic agent identified as effectively modifying EMT in the screening methodologies of the present invention.

A related embodiment is a method of treating a subject suffering from a disorder for which modulation of the MET and/or EMT activity of a cell is desirable. The method comprises administering to the subject an effective amount of a therapeutic agent identified as effectively modifying MET and/or EMT activity in the screening methodologies of the present invention to modulate of the MET and/or EMT activity of a cell in the treated subject.

Yet another embodiment of the invention is the use of the screening methodologies of the present invention to identify a cancer patient that is likely to respond to a known anti-cancer therapy by testing a cancer cell isolated from the patient using the screening methodologies of the present invention. A related embodiment includes the use of the screening methodologies of the present invention to identify a cancer patient that is unlikely to respond to a known anti-cancer therapy by testing a cancer cell isolated from the patient using the screening methodologies of the present invention.

Each publication or patent cited herein is incorporated herein by reference in its entirety. The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention.

### EXAMPLES

### Example 1

### Optimization of 3D-Spheroid Culture for High Throughput Screening (HTS)

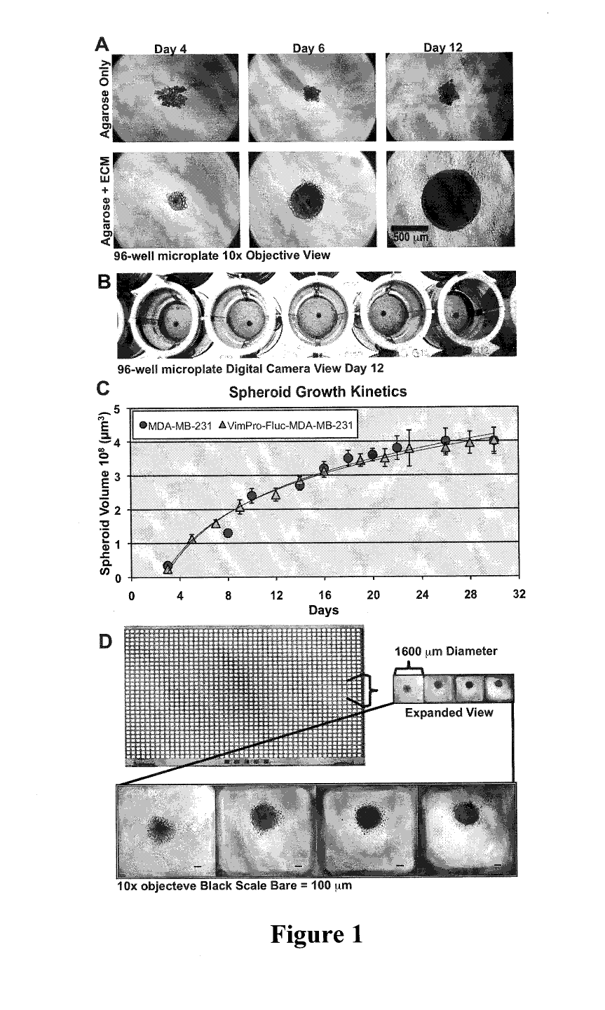

Conventional 3D-spheroid culture utilizes a mixture of laminin/collagen proteins (MATRIGEL™) as a substrate to grow spheroids and recapitulate the tumor microenvironment. However, this methodology typically results in hundreds of spheroids and does not allow for the growth of single uniform spheroids. To circumvent this problem, the inventors have developed a method that combines both agarose, as a non-adherent surface, to coat multi-well plates facilitating tumor cell aggregation (FIG. 1A), and MATRIGEL™ to induce spheroid formation that is not achieved with agarose alone (FIG. 1B-D). Characterization of the spheroid growth kinetics demonstrates a uniform growth rate over 30 days (FIG. 1C). Under these conditions spheroid growth can be accurately controlled ensuring uniformity of spheroids in 96-well plates (FIG. 1B). This methodology has been adapted to obtain uniform single spheroids cultured in 1536-well HTS plates (FIG. 1D). Thus, miniaturizing this model form 96- to 1536-well plates is plausible in order to make the 3D-spheroid models of the present invention suitable for ultra HTS/HCS, and affordable.

### Example 2

### Development of an EMT Biomarker Signature to Monitor EMT/MET

Vimentin (mesenchymal marker): The PGL3-VimPro-Fluc luciferase reporter plasmid was obtained from Dr. Christine Gilles at the University of Liege, Belgium. The VimPro-Fluc cassette was removed and cloned into a lentiviral vector plasmid (pCDH1-CMV-MCS1-EF1-puro), which contains a puromycin resistance gene. Restriction endonucleases were used to digest the DNA flanking both the 5′ (KpnI followed by blunt ending) and 3′ (XbaI) side of the VimPro-Fluc cassette. Compatible digestion sites were not possible on both the 5′ ends of the PGL3 vector or the pCDH1-CMV-MCS1-EF1-puro vector; thus, the non-compatible (ClaI) end was converted to a blunt end. This fragment was purified from a 0.8% agarose gel and ligated into the lentiviral pCDH1-CMV-MCS1-EF1-puro vector replacing the CMV promoter creating pCDH1-VimPro-Fluc-EF1-puro. After ligation, the plasmid was amplified using chemical transformation into DH5α E. coli competent cells and then purified.

Stable cell lines were engineered with both PMC42-LA and MDA-MB-231 cells. Human 293T cells cultured in Dulbecco's Modified Eagle Medium (DMEM)+10% fetal bovine serum (FBS) were seeded at 7.5×106 cells in T-75 flasks for 24 h prior to transfection (to be 70% confluent on day of transfection). Plasmids (pCMV-VSV-G, pHR −8.2 ΔR, and pCDH1-VimPro-Fluc-EF1-puro lentiviral vector) were transfected using liposomal LT 1 transfection reagent. Transfected cells were incubated (37° C., 5% CO₂) for 48 h. The resulting supernatant containing virus was collected and filtered through a 0.45-μm filter and supplemented with polybrene (8 μg/mL). The supernatant containing viral particles was added to MDA-MB-231 or PMC42-LA cells (50% confluent) in 100 mm culture dishes and incubated for 24 h. The medium was removed and then replaced with fresh medium (RPMI-1640+10% FBS). After 3 days, transduced MDA-MB-231 and PMC42-LA cells were selected with 1 μg/mL or 0.5 μg/mL of puromycin, respectively (previously determined with kill curves) over 7 days.

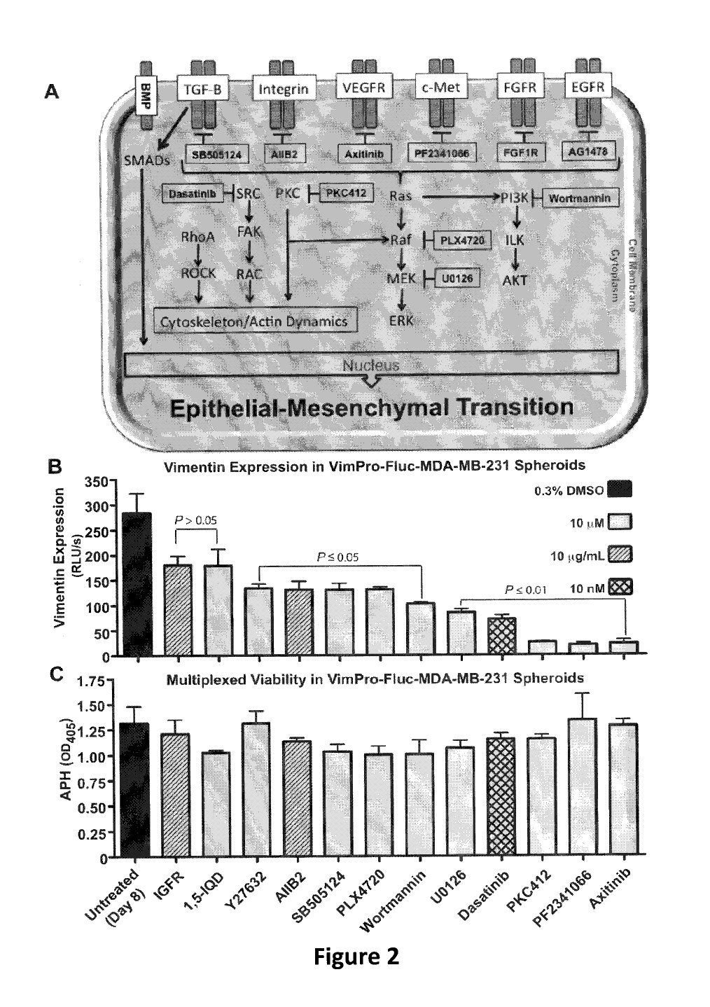

A 2D-screen was conducted with small molecule antitumor agents, growth factors or antibodies (FIG. 2B). All cells were cultured in 100 mm tissue culture dishes in RPMI-1640 supplemented with 10% FBS and maintained in a 5% CO₂ incubator at 37° C. VimPro-Fluc-MDA-MB-231 and VimPro-Fluc-PMC42-LA were seeded at 5,000 cells/well into 96-well white plates and incubated overnight for 16 h. VimPro-Fluc-PMC42-LA and VimPro-Fluc-MDA-MB-231 cells were treated for 72 h. The treatment medium was replaced with 50 μL fresh medium, then 50 μL of One-Glo Luciferase reagent and incubated at room temperature for 10 min. Luminescence was recorded with an integration time of 1 sec/well using a luminescence microplate reader.

Cell lines were cultured as monolayers in 96-well microplates and treated with U0126, the specific inhibitor of mitogen-activated protein kinase kinase (MEK1). VimPro-Fluc-PMC42-LA cells were induced through EMT with 10 ng/mL epidermal growth factor (EGF) treatment for 72 h resulting in upregulation of vimentin expression with concomitant downregulation of E-cadherin. EGF-induced EMT was significantly inhibited by U0126 (P≦0.001) as well as the control AG1478, an epidermal growth factor receptor (EGFR) kinase domain inhibitor. VimPro-Fluc-MDA-MB-231 cells, which highly express vimentin and do not express E-cadherin, showed significant downregulation of vimentin expression after treatment with U0126. However, AG1478 had no statistically significant effect on vimentin expression (P>0.05). Importantly, downregulation of vimentin gene expression correlated with the downregulation of vimentin protein expression in both PMC42-LA and MDA-MB-231 cells. Finally, both MDA-MB-231 and EGF-treated PMC42-LA cells expressing vimentin displayed a mesenchymal phenotype marked by a high invasive potential and this invasive potential was significantly diminished after U0126 treatment. Taken together, this indicates the VimPro-Fluc gene stably integrated in MDA-MB-231 and PMC42-LA cells offers a simplified system in which readily measured and quantitated luciferase expression acts as a surrogate for vimentin expression and EMT.

A 3D-screen was also conducted with small molecule antitumor agents, growth factors or antibodies (FIG. 2A). Uniform single-spheroids of MDA-MB-231 and VimPro-Fluc-MDA-MB-231 breast carcinoma cells were cultured in 96-well flat bottom plates coated with 50 μL of a 1.5% agarose (weight/volume) solution in serum free RPMI-1640 (no phenol red) medium (freshly autoclaved at 121° C. for 15 min). During the coating process the agarose/RPMI-1640 solution was maintained at >60° C. followed by cooling and setting at room temperature for 40 min. Cells were plated at a density of 10,000 cells/well in 50 μL of RPMI-1640 (10% FBS, no phenol red) and centrifuged at 1,000 rpm for 15 min to induce cell aggregation. The aggregated cells were coated with 50 μL of a 10% solution of growth factor reduced MATRIGEL™ (protein concentration: 7.1 mg/mL) in cell culture medium, resulting in a final volume of 100 μL with 5% MATRIGEL™ (laminin/collagen proteins). Spheroids were cultured for 4 days to reach an average diameter of 500 μm under standard tissue culture conditions (37° C., 5% CO₂) without changing or adding medium.

The following known modulators of EMT were purchased: Axitinib and PF-2341066 (Selleck; Houston, Tex.); Dasatinib and PKC412 (LC Laboratory; Woburn, Mass.); PLX4720, (EMD Chemicals; San Diego, Calif.); U0126 (Upstate Cell Signaling Solutions; Lake Placid, N.Y.); 1,5-isoquinolinediol, SB-505124, Wortmannin and Y-27632 (Sigma-Aldrich; St. Louis, Mo.), AIIB2 blocking antibody (University of Iowa Developmental Studies Hybridoma Bank; Iowa City, Iowa) and anti-human IGFIR blocking antibody (R&D Systems; Minneapolis, Minn.). Spheroids were cultured as described above in the appropriate 96-well microplates for 4 days. On day 5 (average spheroid diameter ˜500 μm) the spheroids were treated with 15 μL aliquots of the modulators in RPMI-1640, resulting in a final concentration/well as follows: 10 μM U0126, Axitinib, Y-27632, PLX4720, Wortmannin, SB-505124, PKC412, 1,5-isoquinolinediol, PF-2341066; 1 and 10 nM Dasatinib; and 10 μg/mL IGFIR and AIIB2.

VimPro-Fluc-MDA-MB-231 spheroids were cultured in 96-well optical bottom white plates and treated as described above. On day 8, after 72 h treatment with control EMT modulators, live spheroid vimentin expression was measured by adding 10 μL (final 12.5 μM) of DMNPE-caged luciferin to each well and incubating at 37° C., 5% CO₂ for 2 h.

The inhibition of vimentin was confirmed with qPCR and Western blot. Confocal 3D imaging emphasizes a surface edge of MDA-MB-231 spheroids untreated or treated with (10 μM) U0126 for 72 hours. Using immunohistochemical analysis (DAPI nuclei staining) these images depict the overall vimentin downregulation in MDA-MB-231 spheroids.

For spheroid staining and sectioning, spheroids were cultured and treated with U0126 and Axitinib for 72 h, and transferred into a new 96-well plate, and washed with PBS 2×. Spheroids were fixed with 50 μL of 3.7% formaldehyde for 10 min at room temperature. They were then washed once with PBS and 50 μL of 0.2% Triton X-100 in PBS (v/v) were added for 10 min at room temperature to permeate cells. Next they were blocked with 50 μL of cell culture medium (10% FBS) for 1 h at room temperature. Blocking medium was then aspirated and sphroids were treated with mouse anti-vimentin at 1:100 in TBST containing 20% cell culture medium (v/v) with shaking at 4° C. overnight. They were then washed (3×TBST) and stained with FITC green fluorescent conjugated goat anti-mouse secondary antibody at 1:1,000 and with DAPI (4′,6-diamidino-2-phenylindole) at 1:1,000 in TBST containing 20% cell culture medium (v/v) for 1 h at room temperature and washed with TBST 4×. After fixing with formaldehyde, some spheroids were sectioned using a Leica CM 1950 cryostat (Leica Microsystems; Germany) with thickness of 10 μm. Spheroid sections were washed with PBS and stained as described above. Spheroid sections on slides were covered with mounting medium and cover slips. Imaging of whole spheroids was done with an UltraVIEW® VoX 3D confocal microscope (Perkin Elmer) using a 20× objective. Spheroid sections were imaged using a Nikon D-Eclipse Cl confocal microscope system (Nikon Instruments Inc; Melville, N.Y.).

Vimentin expression in VimPro-Fluc-MDA-MB-231 spheroids was further assessed by immunofluorescent staining of whole spheroids treated with U0126. Significant downregulation of vimentin protein expression was observed throughout the outer surface of U0126 treated spheroids as visualized by 3D confocal microscopy. To measure the extent of penetration into the spheroid by U0126 or Axitinib, spheroids were sectioned and stained for vimentin. These results demonstrate that both U0126 and Axitinib penetrated into the spheroid resulting in downregulation of vimentin throughout the whole spheroid by more than 80% as compared to untreated spheroids.

For 3D-spheroid Western blot analysis, protein was obtained by using 10 spheroids per sample group. The spheroids were collected in 1.5 mL tubes, centrifuged at 10,000 rpm for 5 min at 4° C., and then washed with ice cold PBS (2×). The spheroids were lysed with 66.6 μL spheroid lysis buffer containing 50 mM Tris (pH=8.0), 150 mM NaCl, 1% Nonidet P-40, 20 mM NaF, 2 mM sodium orthovanadate, 20 mM β-glycerophosphate, 1 μg/mL antipain, 1 μg/mL aprotinin, 10 μg/mL leupeptin, 1 μg/mL pepstatin A, and 20 μg/mL phenylmethylsulfonyl fluoride (PMSF). Following lysis each sample group was treated with 33.4 μL of 3× loading buffer (no β-mercaptoethanol). The protein content (10 μL) equivalent of one spheroid for each sample group was subjected to SDS-polyacrylamide gel electrophoresis. Subsequently, protein was transferred to Immobilon-P membranes (Millipore) in transfer buffer (50 mmol/L Tris base, 40 mmol/L glycine, 0.04% sodium dodecyl sulfate, 10% methanol) using an Owl semidry transfer apparatus. The membranes were blocked in 5% non-fat dry milk-TBST (50 mM Tris HCl, 150 mM NaCl, 0.05% Tween-20 (pH=8.0)) and treated with mouse anti-vimentin at 1:1,000 while shaken at 4° C. overnight. Membranes were then washed (3×TBST), probed with anti-mouse-HRP-conjugated secondary antibody (1:10,000 for 1 h at RT), washed again as before and developed with ECL Plus (Amersham; Pittsburgh, Pa.).

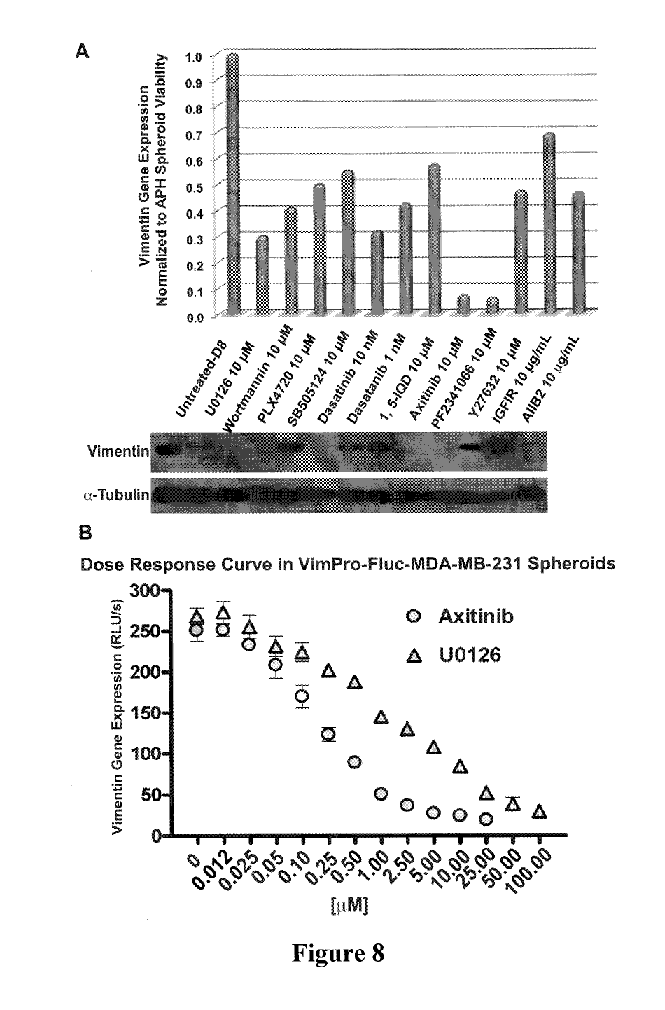

The screening results indicate that a number of these inhibitors downregulate vimentin promoter expression, most notably, Desatanib (10 nM) (FIG. 2B,C). VimPro-Fluc activity was normalized to spheroid viability and compared to vimentin protein expression (FIG. 8A). Based on these data, there appears to be a threshold of 50% downregulation of VimPro-Fluc activity that must be reached before protein downregulation occurs. When VimPro-Fluc activity is downregulated between 50-70% there is significant downregulation of vimentin protein expression. When VimPro-Fluc downregulation reaches ≧70% then downregulation of vimentin protein expression is more pronounced. Of the modulators tested U0126, Dasatinib, Axitinib, and PF2341066 all downregulated VimPro-Fluc activity by ≧70%, which correlated with a significant downreglation of vimentin protein expression. Finally, dose response curves were generated with both U0126 (IC50=2.5 μM) and Axitinib (0.25 μM) (FIG. 8B) demonstrating that these small molecules modulate vimentin expression in a dose dependent manner indicating that their respective target(s) and signaling pathways play a significant role in maintaining vimentin expression and possibly mesenchymal homeostasis. Taken together these data demonstrate that vimentin is an excellent readout to monitor changes in the mesenchymal phenotype during EMT/MET.

### Example 3

### E-Cadherin (Adherence Marker) and ZEB1 (Transcriptional Marker)

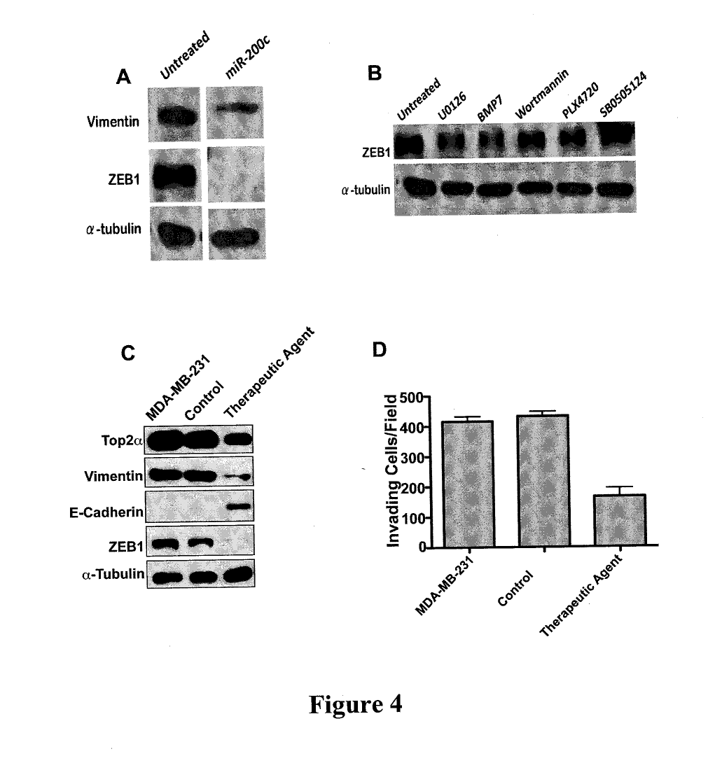

Analysis of both ZEB1 and E-cadherin expression using the methodologies of the present invention resulted in the characterization of these proteins as complementary biomarkers of EMT. While others have demonstrated that downregulation of ZEB1 with miRNA-200C in MDA-MB-231 cells results in partial reversion of EMT with upregulation of E-cadherin (Cochrane, D. R.; Spoelstra, N. S.; Howe, E. N.; Nordeen, S. K.; Richer, J. K., MicroRNA-200c mitigates invasiveness and restores sensitivity to microtubule-targeting chemotherapeutic agents. Mol. Cancer. Ther. 2009), the assays of the present invention were used to characterize a full reversion of EMT or MET using biomarker expression. Delivering mi-R 200c represses ZEB1 expression with concomitant restoration of E-cadherin in multiple cancer cell types (FIG. 4). Furthermore, when ZEB1 expression is repressed with miR-200c, downregulation of vimentin protein expression is also observed (FIG. 4A). ZEB1 protein expression downregulation was also observed when MDA-MB-231 cells were treated with small molecule antitumor agents (FIGS. 4B, 4C). These data demonstrate that MET is characterized by downregulation of the mesenchymal markers vimentin and ZEB1 expression with concomitant upregulation of E-canderin. Importantly, this MET correlates with the inhibition of the invasive potential of metastatic breast cancer cells (FIG. 4D). These data link together each of these proposed biomarkers and support the rationale for choosing this EMT biomarker signature as a useful readout to monitor phenotypic changes in EMT.

### Example 4

### Counter Assays: Multiplexing Cell Viability and Secondary Assay Development

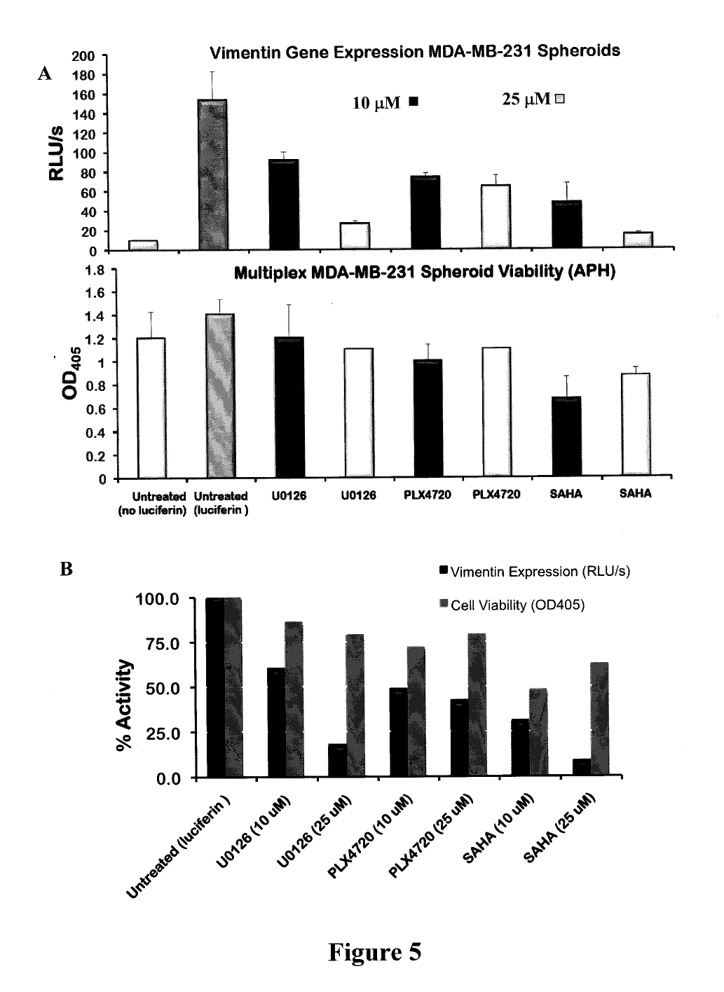

Cell Viability To support the primary assay hit validation, two cell viability assays have been developed: the acid phosphatase assay (APH) and the cell-titer-glow (Promega®). Both assays give robust reproducible results in both 2D and 3D cell culture. We can also multiplex these assays using live VimPro-Fluc stable 3D-spheroids (FIG. 5). Briefly, spheroids were cultured for 4 days and treated on day 5, for 3 days, using the treatments listed in FIG. 5A. On day 8, vimentin expression was measured (luminescence as RLU/s), using a live cell permeable luciferin reagent, followed by multiplexing with the APH assay (OD₄₀₅). Compounds were found that downregulate vimentin expression effectively in 3D, which is not a result of spheroid toxicity (FIG. 5B). Additionally, control modulators of EMT showed no statistically significant changes in spheroid viability (P>0.05) compared to untreated spheroids on day 8. Thus, downregulation of VimPro-Fluc activity by the control modulators is not due to spheroid death. These data demonstrate the multiplexing of cell viability with a live spheroid based assay.

Secondary assay development: As a secondary assay to confirm hits from the primary assay, the Boyden chamber cell invasion method was used and modified to measure the invasive potential of cells isolated from spheroids after treatment with control modulators of EMT. Spheroids were treated as in the primary assay. Following treatment, each sample group was disassociated into single cell suspensions and seeded into invasion chambers followed by a 24 h incubation period. VimPro-Fluc-MDA-MB-231 spheroids were treated with control EMT modulators as described using 15 spheroids per sample group. The spheroids for each sample group were pooled into 1.5 ml, micro tubes and collected by centrifugation at 10,000 rpm for 10 min at room temperature.

Each sample group was then treated with 200 μL of spheroid dissociation solution (0.25% trypsin-EDTA) and incubated at 37° C. for 30 min. Single cell suspensions of each group in RPMI-1640 medium containing 0.1% BSA were seeded at 35,000 cells/chamber (50 μL) into the top chambers of a 96-well invasion plate (BD Biosciences; Bedford, Mass.) previously coated with 50 μL of MATRIGEL™ (40 μg/mL protein in RPMI-1640 medium). Chemoattractant (10%

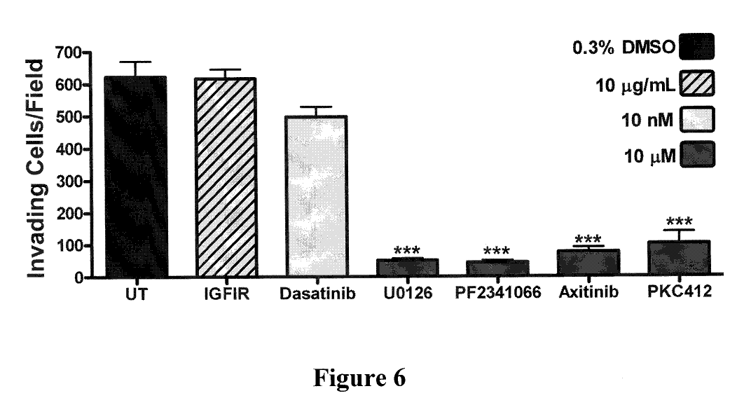

FBS in RPMI-1640 medium, 150 μL) was added to each bottom chamber and the plate was incubated (37° C., 5% CO₂) for 24 h. Following incubation the insert was removed and the medium aspirated from both top and bottom chambers. Cells that did not invade were carefully removed from the top chamber using a cotton swab. Each side of the membrane insert was washed 2× by adding 200 μL PBS-CMF (Ca/Mg free) to the bottom chamber, replacing the insert and adding 50 μL PBS-CMF to the top chamber. The PBS-CMF was removed and the invading cells were fixed with 200 μL of 4% paraformaldahyde in the bottom chambers at 37° C. for 15 min. The insert was washed 2× with PBS-CMF as described above. The invading cells were subsequently stained with 200 μL of DAPI (3 μg/mL) in PBS-CMF for 30 min at room temperature and washed 4×, as described above, in 15 min intervals. Invading cells were quantified using five random fields per filter insert in triplicate at 20× magnification using a fluorescent microscope equipped with metamorph image analysis and quantification software (Molecular Devices; Downingtown, Pa.). Under these conditions the control modulators IGFIR, Dasatinib, U0126, PF2341066, Axitinib, and PKC412 were tested. U0126, PF2341066, Axitinib and PKC412 caused significant inhibition of the invasive potential of MDA-MB-231 spheroids. Conversely, Dasatinib, a potent inhibitor of vimentin gene expression, did not significantly alter the invasive potential of MDA-MB-231 spheroids (FIG. 6). These data indicate that cell invasion is a functional endpoint that can be used as a complementary secondary assay to validate hits and rule out false positive hits identified in the primary screen.

### Example 5

### Single EMT Marker and Cell Viability Multiplex Analysis

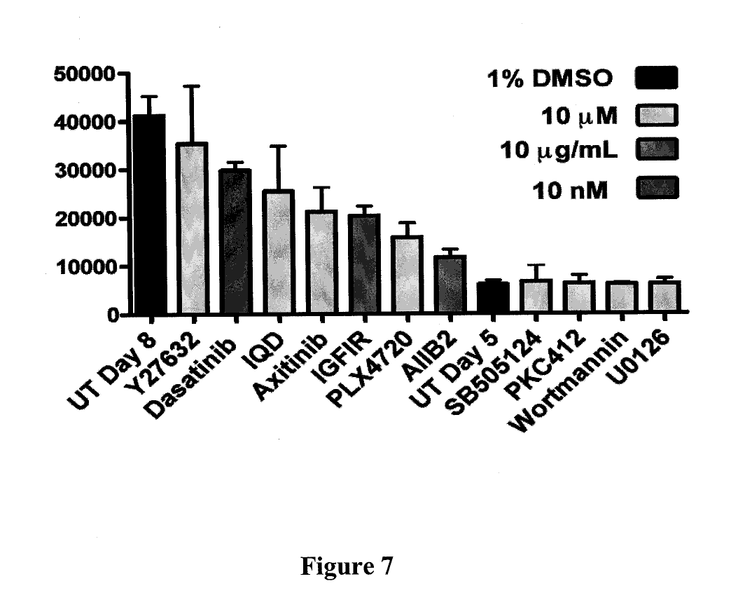

Vimentin expression was analyzed using the screening assays of the present invention in conjunction with cell viability assays to evaluate potential therapeutic agents. Vimentin expression was measured in live breast cancer cells (MDA-MB-231) growing as cell spheroids in 96-well plates following treatment with 11 potential therapeutic agents (FIG. 7). By measuring cell viability and comparing Vimentin expression, agents are identified that suppress Vimentin expression, compared to control, while the spheroid cells remain viable. (FIG. 8A). Dose response curves for two of these agents, which modulate EMT (U0126 and Axitinib) are shown in FIG. 8B.

### Example 6

### Immunofluoroescence Analysis of Breast Cancer Cell Spheroids

In a cell spheroid, as in a solid tumor, the inner core is predominately necrotic tissue, while the peripheral layer of cells forms the primary zone of cell proliferation (Lin, R. Z. and Chang, H. Y. Recent Advances in Three-Dimensional Multicellular Spheroid Culture for Biomedical Research. Biotechno. J. 2008, 3(9-10):1172-84). Vimentin expression was visualized by immunofluorescence analysis in a spheroid of breast cancer cells (MDA-MB-231) before and following treatment with Dasatinib and U0126. Immunofluorescence shows repression of Vimentin expression in the peripheral layer of spheroid cells following treatment with these agents.

### Example 7

### Marine Natural Products Pilot Screening

To validate that this 3D-spheroid model is suitable for HTS a pilot screen was conducted with a marine natural product library of 230 pure compounds. The marine natural products library compounds were formatted into 3 mM stock solutions in DMSO and screened using a final concentration of 10 μM with a final volume of 0.3% DMSO. These compounds were screened beside 10 μM Axitinib, a positive control that downregulates vimentin expression, and 0.3% DMSO as negative control and vehicle % used that does not modulate vimentin expression.

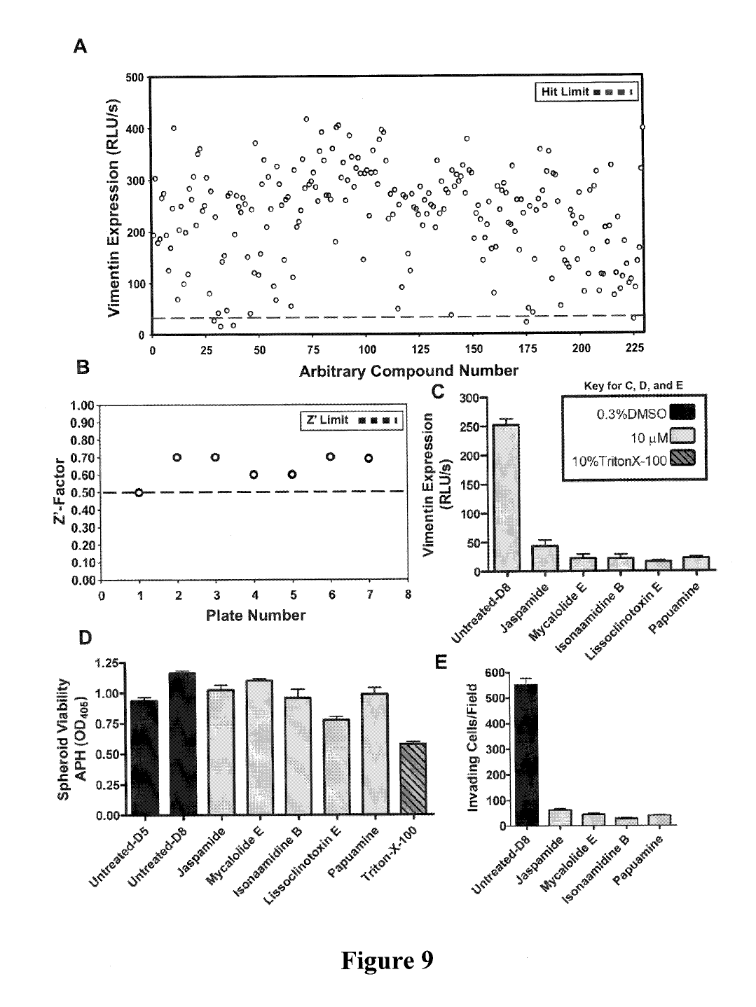

Uniform single spheroids were cultured in 96-well optical bottom white plates as described above and treated on day 5. To avoid edge effects the outer wells were unused and filled with sterilized water. 15 μL of controls and marine natural products were added to each well at final concentrations described above. After 72 h, vimentin expression was measured by adding 10 μL (final 12.5 μM) of DMNPE-caged luciferin to each well and incubated for 2 h (37° C., 5% CO₂). Luminescence was recorded with an integration time of 1 second/well (FIG. 9A-D). Directly following the primary assay measuring vimentin expression, spheroid viability was multiplexed using the APH assay. Hits were confirmed using the cell invasion assay (FIG. 9E).

Data were subjected to unpaired one-tailed t-test statistical analysis using Prism (Graphpad Software; San Diego, Calif.) and P values ≦0.01 or 0.001 were considered statistically significant. Fluorescence intensity and Western blot quantification was carried out using ImageJ software (National Institutes of Health; Bethesda, Md.). The marine natural product pilot screen and 3D-spheroid assay were validated using the statistical parameter Z′-factor.

Using the Axitinib positive control and the DMSO negative control, the Z′-factor analysis was used to validate the pilot screen. Z′ measures how robust an assay is based on comparison of the relative means and standard deviations of the positive and negative controls. We calculated the average Z′-factor per plate to be 0.64, which is considered to be an excellent assay. Other supporting statistical data including signal to noise (S/N), signal to background (S/B) and % coefficient of variance (CV) are tabulated in the supplementary data section. From the 230 compounds screened, using three standard deviations away from the mean as a hit limit (RLU/s=35), we identified 5 hits that downregulated vimentin expression by about 90% and were below the calculated hit limit. We then rescreened these hits in triplicate, which gave consistent results and statistical validation. The downregulation of vimentin expression by these hits was not due to cell death as compared to our control Triton-X-100, which significantly induced spheroid death. However, as compared to day 5 untreated spheroids, a hit identified as lissoclinotoxin E21, displayed a statistically significant decrease in spheroid viability (P<0.01). Therefore, lissoclinotoxin E was considered to be a false positive hit.

After examining the remaining hits from the primary pilot screen we identified two compounds with known mechanisms of action and two hits with no known molecular targets. These hits were then confirmed by secondary assay screening.

The foregoing description of the present invention has been presented for purposes of illustration and description. Furthermore, the description is not intended to limit the invention to the form disclosed herein. Consequently, variations and modifications commensurate with the above teachings, and the skill or knowledge of the relevant art, are within the scope of the present invention. The embodiments described hereinabove are further intended to explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

What is claimed is:

1. A method of screening agents for activity in modulating the EMT phenotype of a cell comprising:
a. establishing at least one three-dimensional (3D) spheroid of cells in individual wells of a multi-welled plate that is capable of sustaining a tissue culture
b. contacting the cell spheroid(s) with a potential therapeutic agent
c. assaying for a marker indicative of modulation of EMT activity.

2. The method of claim 1, wherein the cells are cells capable of sustained growth under tissue culture conditions.

3. The method of claim 1, wherein the cells are neoplastic cells.

4. The method of claim 1, wherein the cells are tumor cells that have been isolated from a human.

5. The method of claim 1, wherein the cells are human malignant tumor cells selected from the group consisting of breast cancer, lung cancer, prostate cancer, colon cancer, melanoma cancer, and cancer of the bone and connective tissues.

6. The method of claim 1, wherein the cells are stem cells selected from the group consisting of embryonic stem cells or adult stem cells, progenitor cells, bone marrow stromal cells macrophages, fibroblast cells, endothelial cells, epithelial cells, and mesenchymal cells.

7. The method of claim 1, wherein the multi-welled plate is a 96-well cell culture plate.

8. The method of claim 1, wherein the multi-welled plate is a 1536-well cell culture plate.

9. The method of claim 1, wherein the multi-welled plate is coated with collagen IV basement membrane.

10. The method of claim 1, wherein the multi-welled plate is coated with agarose.

11. The method of claim 1, wherein the multi-welled plate is coated with collagen IV basement membrane and agarose.

12. The method of claim 1, wherein the potential therapeutic agent is at least one of:
a. a molecule selected from the group consisting of an endogenous ligand or ligands, a biological sample suspected of containing a native or endogenous ligand or ligands, a combinatorial library of small molecules, a hormone, an antibody, a polysaccharide, an anti-cancer agent, a natural product, a terrestrial product and a marine natural product;
b. a molecule that binds with high affinity to a biopolymer selected from the group consisting of a protein, a nucleic acid, and a polysaccharide; and,
c. a purified biological molecule selected from the group consisting of a protein, a nucleic acid, a silencing RNA (siRNA), a micro RNA (miRNA), and a short hairpin RNA (shRNA).

13. The method of claim 1, wherein the cells forming the spheroid(s) of cells, are transformed with at least one heterologous nucleic acid molecule that encodes one or more biomarkers associated with the epithelial or mesenchymal phenotypes.

14. The method of claim 13, wherein the recombinant nucleic acid molecule(s) are chromosomally integrated into the genome of the cell.

15. The method of claim 13, wherein the biomarkers are linked to an indicator that can be detected in situ following expression of the biomarker.

16. The method of claim 15, wherein the indicator is a compound that is readily detectable using a detection technique selected from the group consisting of dark versus light detection, fluorescence or chemiluminescence spectrophotometry, scintillation spectroscopy, chromatography, liquid chromatography/mass spectroscopy (LC/MS), and colorimetry.

17. The method of claim 15, wherein the indicator compound is at least one of fluorogenic or fluorescent compound, chemiluminescent compound, calorimetric compound, UV/VIS absorbing compound, radionucleotide, Red Fluorescence Protein (RFP), Green Fluorescent Protein (GFP), luciferase, and combinations thereof.

18. The method of claim 1, wherein the marker is at least one of Epithelial (E)-cadherin, Zinc finger E-box binding homeobox 1 (ZEB1), and Vimentin.

19. The method of claim 1, further comprising:
determining the functional effect of the potential therapeutic agent on migration of at least one cell dissociated from the cell spheroid(s) through a collagen-coated membrane.

20. A method to select a cancer patient who is predicted to benefit or not benefit from therapeutic administration of an inhibitor of EMT, comprising:
a) establishing at least one three-dimensional (3D) spheroid of cells from a sample of tumor cells from a patient a level of a biomarker selected from the group consisting of Epithelial (E)-cadherin, Zinc finger E-box binding homeobox 1 (ZEB1), and Vimentin;
b) comparing the level of the biomarker in the tumor cell sample to a control level of the biomarker selected from the group consisting of:
i) a control level of the biomarker that has been correlated with sensitivity to the inhibitor; and
ii) a control level of the biomarker that has been correlated with resistance to the inhibitor; and
c) selecting the patient as being predicted to benefit from therapeutic administration of the inhibitor, if the level of the biomarker in the patient's tumor cells is statistically similar to or greater than the control level of the biomarker that has been correlated with sensitivity to the inhibitor, or if the level of the biomarker in the patient's tumor cells is statistically greater than the level of the biomarker that has been correlated with resistance to the inhibitor; or
d) selecting the patient as being predicted to not benefit from therapeutic administration of the inhibitor, if the level of the biomarker in the patient's tumor cells is statistically less than the control level of the biomarker that has been correlated with sensitivity to the inhibitor, or if the level of the biomarker in the patient's tumor cells is statistically similar to or less than the level of the biomarker that has been correlated with resistance to the inhibitor.
